SEQUENCE LISTING (27 sequences)

SEQ ID NO: 1 - protein, 8 aa, Homo sapiens
GFTFSSYA

SEQ ID NO: 2 - protein, 8 aa, Homo sapiens
ISGSGGST

SEQ ID NO: 3 - protein, 9 aa, Homo sapiens
AKSYGAFDY

SEQ ID NO: 4 - protein, 116 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSS

SEQ ID NO: 5 - protein, 445 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 445 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 445 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 8 aa, Homo sapiens
SSNIANGI

SEQ ID NO: 9 - skipped

SEQ ID NO: 10 - protein, 10 aa, Homo sapiens
GTWDFSYGIV

SEQ ID NO: 11 - protein, 8 aa, Homo sapiens
SSNIGIGL

SEQ ID NO: 12 - skipped

SEQ ID NO: 13 - protein, 10 aa, Homo sapiens
GTWDFSYRVD

SEQ ID NO: 14 - protein, 109 aa, Homo sapiens
QSVLTQPPSVSAAPGQKVTISCSGSSSNIANGIVSWYQQLPGTAPKLLIYFDNLRPSGIPDRFSGSKSGTSATLGITGLQTGDEADYYCGTWDFSYGIVFGGGTKLTVL

SEQ ID NO: 15 - protein, 109 aa, Homo sapiens
QSVLTQPPSVSAAPGQKVTISCSGSSSNIGIGLVSWYQQLPGTAPKLLIYNVNTRPSGIPDRFSGSKSGTSATLGITGLQTGDEADYYCGTWDFSYRVDFGGGTKLTVL

SEQ ID NO: 16 - protein, 215 aa, Homo sapiens
QSVLTQPPSVSAAPGQKVTISCSGSSSNIANGIVSWYQQLPGTAPKLLIYFDNLRPSGIPDRFSGSKSGTSATLGITGLQTGDEADYYCGTWDFSYGIVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 17 - protein, 215 aa, Homo sapiens
QSVLTQPPSVSAAPGQKVTISCSGSSSNIGIGLVSWYQQLPGTAPKLLIYNVNTRPSGIPDRFSGSKSGTSATLGITGLQTGDEADYYCGTWDFSYRVDFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 12 aa, Homo sapiens
QSVLYSSNNKNY

SEQ ID NO: 19 - skipped

SEQ ID NO: 20 - protein, 9 aa, Homo sapiens
QQNLRPPET

SEQ ID NO: 21 - protein, 12 aa, Homo sapiens
QSVLYSSNNKNY

SEQ ID NO: 22 - skipped

SEQ ID NO: 23 - protein, 9 aa, Homo sapiens
QQNFRPPET

SEQ ID NO: 24 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQNLRPPETFGQGTKVEIK

SEQ ID NO: 25 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVGVYYCQQNFRPPETFGGGTKVEIK

SEQ ID NO: 26 - protein, 220 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQNLRPPETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 220 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVGVYYCQQNFRPPETFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0075000 A1
(43) Pub. Date: Mar. 6, 2025

(54) COMBINATION THERAPY WITH A CEA X CD28 BISPECIFIC ANTIBODY AND BLOCKING ANTI-PD-1 ANTIBODIES FOR ENHANCED IN VIVO ANTI-TUMOR ACTIVITY

(71) Applicant: Novimmune SA, Geneva (CH)

(72) Inventors: Sara MAJOCCHI, Geneva (CH); Limin SHANG, Bellevue (CH); Walter G. FERLIN, Collonges-Sous-Salève (FR)

(21) Appl. No.: 18/825,304

(22) Filed: Sep. 5, 2024

Related U.S. Application Data
(60) Provisional application No. 63/580,889, filed on Sep. 6, 2023.

(51) Int. Cl. C07K 16/30; A61K 39/00; A61P 35/00; C07K 16/28

(52) U.S. Cl. CPC C07K 16/3007; A61P 35/00; C07K 16/2818; A61K 2039/505; C07K 2317/24; C07K 2317/31

(57) ABSTRACT

The invention provides a CEAxCD28 bispecific antibody for the CEA-targeted costimulation of tumor infiltrating T cells in combination with antibodies which block the PD-L1/PD-1 pathway, for use in the combination treatment for stimulating an immune response against cancer or tumor.

### RELATED APPLICATIONS

This claims the benefit of U.S. Provisional Application No. 63/580,889, filed on Sep. 6, 2023, and is incorporated herein by reference in its entirety.

### INCORPORATION BY REFERENCE OF SEQUENCE LISTING

The contents of the electronic sequence listing (NOVI_053_001US_SeqList_ST26.xml; Size: 24,114 bytes; and Date of Creation: Sep. 6, 2023) are herein incorporated by reference in its entirety.

### FIELD

The present invention relates generally particular to the use of CEAxCD28 bispecific antibodies in combination with a PD-1 blocking antibody for treating cancers

### BACKGROUND OF THE INVENTION

In the past years, novel approaches to stimulate the body's own immune cells to better attack and kill cancer cells have been developed. Approved cancer immunotherapies include T cell bispecific antibodies—bridging T cells to target cells expressing a tumor associated antigen (TAA), via the CD3 receptor on T cells and on Chimeric Antigen Receptor (CAR) T cells. Despite the very good anti-tumor responses observed with treatments using T cell bispecific antibodies or CAR T cells in hematological malignancies, there have been no significant breakthroughs of these approaches in the context of solid cancers to date, leaving many cancer patients with no therapeutic options.

Therefore, and in spite of the success of these immunotherapies in some cancer types, a large fraction of cancer patients lacks valid therapeutic options, highlighting the need for new treatments. The use of molecules capable of activating the immune system by targeting costimulatory signals on T cells open the way to novel therapeutic options for solid cancer patients.

A need exists for compositions and methods for targeting T-cell activation useful for treating solid cancers. Provided herein are methods and compositions addressing this need.

### SUMMARY OF THE INVENTION

In some aspects, the techniques described herein relate to a method of treating a cancer in a subject including administering to the subject a CEAxCD28 bispecific antibody and a blocking anti-PD-1 antibody.

In some aspects, the techniques described herein relate to a method, wherein the CEAxCD28 bispecific antibody includes a. a CEA antigen binding domain including: i. a heavy chain variable region having a complementarity determining region 1 (CDRH1) including the amino acid sequence of (SEQ ID NO: 1); a complementarity determining region 2 (CDRH2) including the amino acid sequence of (SEQ ID NO: 2); and a complementarity determining region 3 (CDRH3) including the amino acid sequence of (SEQ ID NO: 3); and ii. a first light chain variable region having a complementarity determining region 1 (CDRL1) including the amino acid sequence of SEQ ID NO: 11; a complementarity determining region 2 (CDRL2) including the amino acid sequence of SEQ ID NO: 12; and a complementarity determining region 3 (CDRL3) including the amino acid sequence of SEQ ID NO: 13; b. a CD28 binding domain including: i. a heavy chain variable region having a complementarity determining region 1 (CDRH1) including the amino acid sequence of (SEQ ID NO: 1); a complementarity determining region 2 (CDRH2) including the amino acid sequence of (SEQ ID NO: 2); and a complementarity determining region 3 (CDRH3) including the amino acid sequence of (SEQ ID NO: 3); and ii. a first light chain variable region having a complementarity determining region 1 (CDRL1) including the amino acid sequence of SEQ ID NO: 18; a complementarity determining region 2 (CDRL2) including the amino acid sequence of SEQ ID NO: 19; and a complementarity determining region 3 (CDRL3) including the amino acid sequence of SEQ ID NO: 20.

In some aspects, CEA antigen binding domain include a variable light chain including the amino acid sequence of SEQ ID: 15; and a variable heavy chain including the amino acid sequence of SEQ ID: 4; and the CD28 antigen binding domain include a variable light chain including the amino acid sequence of SEQ ID: 24; and a variable heavy chain including the amino acid sequence of SEQ ID: 4.

In some aspects, the CEA antigen binding domain include a light chain including the amino acid sequence of SEQ ID: 17; and a variable heavy chain including the amino acid sequence of SEQ ID: 7; and a CD28 antigen binding domain include a variable light chain including the amino acid sequence of SEQ ID: 26; and a variable heavy chain including the amino acid sequence of SEQ ID: 4.

The blocking anti-PD-1 antibody includes for example nivolumab, pembrolizumab, cemiplimab, sintilimab, camrelizumab, toripalimab, tislelizumab, zimberelimab, prolgolimab, and dostarlimab.

The cancer includes for example, leukemias, lymphomas, breast cancer, colon cancer, ovarian cancer, bladder cancer, prostate cancer, glioma, lung & bronchial cancer, colorectal cancer, pancreatic cancer, esophageal cancer, liver cancer, urinary bladder cancer, kidney and renal pelvis cancer, oral cavity & pharynx cancer, uterine corpus cancer, and/or melanoma.

In some aspects, the techniques described herein relate to a method, wherein the bispecific antibody and the anti-PD-1 antibody are administered simultaneously.

In some aspects, the techniques described herein relate to a method, wherein the bispecific antibody is administered prior to anti-PD-1 antibody.

In some aspects, the techniques described herein relate to a method, wherein the bispecific antibody is administered after the anti-PD-1 antibody.

In some aspects, the techniques described herein relate to a method, wherein the CEAxCD28 bispecific antibody and/or the blocking anti-PD-1 antibody are administered intravenously.

The subject is human. The subject is known to or suspected of having cancer.

The treatment methods described herein reduces the proliferation of the cancer cell, increase killing of a cancer cell and/or enables tumor-specific T cell activation.

The administration of the blocking anti-PD-1 increases the anti-tumor activity of the bispecific antibody compared to when the bispecific antibody is administered as a single therapeutic agent.

In some aspects, the techniques described herein relate to a formulation including an CEAxCD28 bispecific antibody and a blocking anti-PD-1 antibody.

In some aspects, the techniques described herein relate to a formulation, wherein the CEAxCD28 bispecific antibody includes a. a CEA antigen binding domain including: i. a heavy chain variable region having a complementarity determining region 1 (CDRH1) including the amino acid sequence of (SEQ ID NO: 1); a complementarity determining region 2 (CDRH2) including the amino acid sequence of (SEQ ID NO: 2); and a complementarity determining region 3 (CDRH3) including the amino acid sequence of (SEQ ID NO: 3); and ii. a first light chain variable region having a complementarity determining region 1 (CDRL1) including the amino acid sequence of SEQ ID NO: 11; a complementarity determining region 2 (CDRL2) including the amino acid sequence of SEQ ID NO: 12; and a complementarity determining region 3 (CDRL3) including the amino acid sequence of SEQ ID NO: 13; b. a CD28 binding domain including: i. a heavy chain variable region having a complementarity determining region 1 (CDRH1) including the amino acid sequence of (SEQ ID NO: 1); a complementarity determining region 2 (CDRH2) including the amino acid sequence of (SEQ ID NO: 2); and a complementarity determining region 3 (CDRH3) including the amino acid sequence of (SEQ ID NO: 3); and ii. a first light chain variable region having a complementarity determining region 1 (CDRL1) including the amino acid sequence of SEQ ID NO: 18; a complementarity determining region 2 (CDRL2) including the amino acid sequence of SEQ ID NO: 19; and a complementarity determining region 3 (CDRL3) including the amino acid sequence of SEQ ID NO: 20; and c. the PD-1 blocking antibody is selected from the group consisting of nivolumab, pembrolizumab, cemiplimab, sintilimab, camrelizumab, toripalimab, tislelizumab, zimberelimab, prolgolimab, and dostarlimab.

In some aspects, the techniques described herein relate to a kit including: 1. a formulation suitable for human administration including a CEAxCD28 bispecific antibody including a. a CEA antigen binding domain including: i. a heavy chain variable region having a complementarity determining region 1 (CDRH1) including the amino acid sequence of (SEQ ID NO: 1); a complementarity determining region 2 (CDRH2) including the amino acid sequence of (SEQ ID NO: 2); and a complementarity determining region 3 (CDRH3) including the amino acid sequence of (SEQ ID NO: 3); and ii. a first light chain variable region having a complementarity determining region 1 (CDRL1) including the amino acid sequence of SEQ ID NO: 11; a complementarity determining region 2 (CDRL2) including the amino acid sequence of SEQ ID NO: 12; and a complementarity determining region 3 (CDRL3) including the amino acid sequence of SEQ ID NO: 13; b. a CD28 binding domain including: i. a heavy chain variable region having a complementarity determining region 1 (CDRH1) including the amino acid sequence of (SEQ ID NO: 1); a complementarity determining region 2 (CDRH2) including the amino acid sequence of (SEQ ID NO: 2); and a complementarity determining region 3 (CDRH3) including the amino acid sequence of (SEQ ID NO: 3); and ii. a first light chain variable region having a complementarity determining region 1 (CDRL1) including the amino acid sequence of SEQ ID NO: 18; a complementarity determining region 2 (CDRL2) including the amino acid sequence of SEQ ID NO: 19; and a complementarity determining region 3 (CDRL3) including the amino acid sequence of SEQ ID NO: 20; and a formulation suitable for human administration including a PD-1 blocking antibody such as nivolumab, pembrolizumab, cemiplimab, sintilimab, camrelizumab, toripalimab, tislelizumab, zimberelimab, prolgolimab, and dostarlimab; and to instructions for use.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1. Shows the mode of action of the combination of CD28 costimulatory bispecific antibodies such as the CEAxCD28 of the present invention with blocking anti-PD-1 antibodies. Blockade of the PD-1 on T cells release the brake on CD28 signaling, while the CD28 bispecific antibody provides costimulatory signal in a CEA-dependent manner to better activate the T cells.

FIG. 2. The experimental design of the in vivo efficacy study shown on FIG. 3, where the CEAxCD28 bispecific antibody AI3AC84/N was tested in combination with a blocking in anti-mouse PD-1 antibody in a syngeneic MC38-huCEA subcutaneous tumor model in CD28 humanized mice. Groups and treatment regimen are reported.

FIGS. 3A-3D. AI3AC84/N synergizes with anti-PD-1 mAb to significantly enhance in vivo anti-tumoral activity. In vivo efficacy study of AI3AC84/N in combination with a blocking anti-mouse PD-1 antibody in the model described in FIG. 2. The average tumor volume (FIG. 3A), the growth of tumors in individual mice (FIG. 3B), the comparison of the tumor volume at day 21 (FIG. 3C) as well as the survival curve (FIG. 3D) are shown.

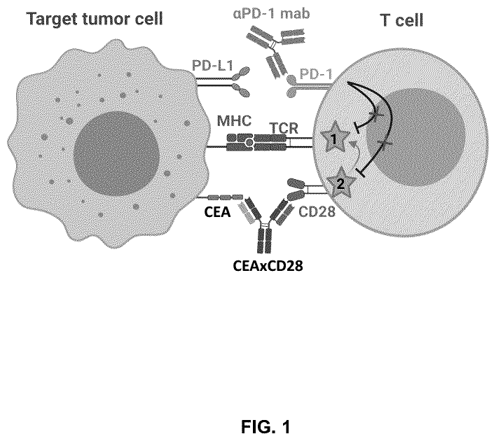

### DETAILED DESCRIPTION OF THE INVENTION

The invention is based in part on a bispecific antibody (bsAb) capable of tumor-dependent T cell activation and tumor cell killing. Specifically, the invention is based upon bsAb co-engagement of carcinoembryonic antigen (CEA) expressed at the surface of the target cells to mediate CD28 clustering and therefore, tumor-specific T cell activation.

The bsAbs of the invention are characterized by a single agonist CD28 antigen binding domain for the monovalent co-stimulation of CD28, and a second antigen binding domain capable of binding specifically and monovalently to CEA for the targeted delivery of the bsAb to the tumor microenvironment. The bsAbs of the invention are referred to herein as CD28xCEA bsAbs or CEAxCD28 bsAbs.

The CEAxCD28 bsAb are administered together with a PD-1 blocking antibody. Administration of the PD-1 blocking antibody can be concurrent or sequential with administration of the CEAxCD28 bsAb. Because PD-1 negatively regulates the immune response, blockade of the PD-1 on T cells results in better T cell activation and proliferation. The T cell costimulatory signal provided by the CD28 bispecific antibody in presence of CEA+ target cells further activates the T cells. As shown herein, the CEAxCD28 bsAb synergizes with anti-PD-1 mAb to significantly enhance in vivo anti-tumoral activity, thanks to the complementary mechanisms of action.

CD28 is a key co-stimulatory receptor expressed at the surface of T lymphocyte. It belongs to a subfamily of costimulatory molecules characterized by an extracellular variable immunoglobulin-like domain. Other members in the family of molecule include CTLA-4, ICOS, PD-1 and BTLA.

In humans, CD28 is expressed at the cell surface of T lymphocyte as a disulfide-linked homodimer and is found on approximately 80% of human CD4+ T cells and 50% of CD8+ T cells.

Despite lacking intrinsic enzymatic activity, CD28 engagement by its ligands leads to specific phosphorylation and transcriptional signaling which ultimately results in metabolic changes and in the production of key cytokines, chemokines, and survival signals that are essential for long-term expansion and differentiation of T cells.

The primary ligands for CD28 are CD80 (B7.1) and CD86 (B7.2), which are mainly expressed at the surface of professional antigen presenting cells (APC). CD80 and CD86 diverge in their expression patterns, multimeric states, and functionality. Because CD28 and CTLA-4 are highly homologous, they compete for the same ligands. However, since CTLA-4 binds these ligands with a higher affinity than CD28, CTLA-4 competes with CD28 for ligands and ultimately suppresses T cells responses.

Several anti-CD28 monoclonal antibodies have been proposed for the therapeutic targeting of CD28. A fraction of the identified anti-CD28 antibodies, termed superagonist (SA) antibodies, were found to induce the full activation of primary resting T cells even in the absence of TCR ligation (signal 1), via the clustering of CD28 at the surface of the T cells. The first-in-human study of one of such SA anti-CD28 antibodies, TGN1412, resulted however in severe inflammatory reactions as well as chronic organ failure in all healthy volunteers subjected to the treatment. A cytokine storm predicted by neither in vivo nor in vitro preclinical safety studies was the cause of these adverse events.

To avoid the safety issues linked to SA antibodies or to systemic CD28 co-stimulation, tumor-targeted CD28 bispecific antibodies can be designed to co-stimulate T cells specifically within the tumor microenvironment. By pairing an agonist anti-CD28 binding domain to a CEA binding domain, molecules capable of bridging T cells to malignant cells expressing CEA are generated. Since CD28 bsAbs can only bind to CD28 monovalently, CD28 cannot be successfully clustered in the absence of CEA-positive target cells, thus preventing systemic T cell activation.

Furthermore, even in presence of CEA-positive cancer cells which allow for CD28 clustering at the surface of the T cells, the full cytotoxic potential of T cells can only be unleashed by CEAxCD28 bispecific antibodies in presence of primary T cell stimulation via the TCR. This contrasts with the bivalent superagonist CD28 monoclonal antibodies that were described above.

### CEAxCD28 Bispecific Antibodies

The bsAbs antibodies according to the invention may be generated de novo or may be engineered from existing monospecific CD28 antibodies and CEA antibodies. The bispecific antibodies of the invention have one antigen binding region that is specific for CD28 and a second antigen binding region that is specific for a CEA. CD28 and CEA antibodies that may be used to engineer bispecific molecules include the antibodies described herein and in PCT/IB2023/000124, the contents of which are incorporated by reference in its entirety. Exemplary anti-CD28 antibodies from which the CD28 antigen binding region can be derived from include the “AI3” or “AI13”, antibody. Exemplary anti-CEA antibodies from which the CEA antigen binding region can be derived from include the “AC22”, or “AC84” antibody.

The bsAbs can be based on any of the different antibody formats that have been previously described. In general, IgG-like formats are preferred as they provide favorable properties such as long half-life and potentially reduced immunogenicity, but any other molecular bispecific format can also be used for the invention. In some embodiments, the bispecific antibodies share a common heavy chain. In some embodiments, the heavy chains are native heavy chains (i.e., does not contain any mutations (“wildtype”)). In some embodiments, the heavy chains comprise at least one mutation (i.e., with “LALA” mutation or “LALAPA” mutation). Optionally, the bispecific antibodies have light chains of different types. For example, one light chain is a kappa light and the other light chain is a lambda light chain (i.e., kl-body) Differing light chains allows the bispecific to be purified easily using kappa and lambda select resins.

The heavy and light chain amino acid sequences of the antibodies identified by their United States Adopted Names (USAN are available for example via the American Medical Association).

Monospecific CD28 and CEA binding variable domains may be selected de novo from for example a phage display library, where the phage is engineered to express human immunoglobulins or portions thereof such as Fabs, single chain variable fragments (scFv), or unpaired or paired antibody variable regions and subsequently engineered into a bispecific format. The CD28 and CEA variable domains can be isolated for example from phage display libraries expressing antibody heavy and light chain variable regions as fusion proteins at the surface of bacteriophage M13, fused to the capsid protein pIII.

The antibody libraries are screened for binding CD28 antibodies and CEA and the obtained positive clones are further characterized. Such phage display methods for isolating human antibodies are established in the art. See for example: U.S. Pat. Nos. 5,223,409; 5,403,484; and 5,571,698, 5,427,908, 5,580,717, 5,969,108, 6, 172, 197, 5,885,793; 6,521,404; 6,544,731; 6,555,313; 6,582,915 and 6,593,081. The obtained de novo variable regions binding are engineered to bispecific formats using the methods know in the art and described herein.

Additionally, bispecific antibodies of the invention can be made using the techniques, including those disclosed in WO 2012/023053, filed Aug. 16, 2011, the contents of which are hereby incorporated by reference in their entirety. The methods described in WO 2012/023053 generate bispecific antibodies that are identical in structure to a human immunoglobulin. This type of molecule is composed of two copies of a unique heavy chain polypeptide, a first light chain variable region fused to a constant Kappa domain and second light chain variable region fused to a constant Lambda domain. Each combining site displays a different antigen specificity to which both the heavy and light chain contribute. The light chain variable regions can be of the Lambda or Kappa family and are preferably fused to a Lambda and Kappa constant domains, respectively. This is preferred in order to avoid the generation of non-natural polypeptide junctions.

However, it is also possible to obtain bispecific antibodies of the invention by fusing a Kappa light chain variable domain to a constant Lambda domain for a first specificity and fusing a Lambda light chain variable domain to a constant Kappa domain for the second specificity. The bispecific antibodies described in WO 2012/023053 are referred to as IgGκλ antibodies or “κλ bodies,” a new fully human bispecific IgG format. This KA-body format allows the affinity purification of a bispecific antibody that is undistinguishable from a standard IgG molecule with characteristics that are undistinguishable from a standard monoclonal antibody and, therefore, favorable as compared to previous formats.

In addition to methods described above, bispecific antibodies can be generated in vitro in a cell-free environment by introducing asymmetrical mutations in the CH3 regions of two monospecific homodimeric antibodies and forming the bispecific heterodimeric antibody from two parent monospecific homodimeric antibodies in reducing conditions to allow disulfide bond isomerization according to methods described in Intl. Pat. Publ. No. WO2011/131746. In the methods, the first monospecific bivalent antibody and the second monospecific bivalent antibody are engineered to have certain substitutions at the CH3 domain that promoter heterodimer stability; the antibodies are incubated together under reducing conditions sufficient to allow the cysteines in the hinge region to undergo disulfide bond isomerization; thereby generating the bispecific antibody by Fab arm exchange.

Antibodies of the present invention have two or more antigen binding domains and are bispecific. Bispecific antibodies of the invention include antibodies having a full length antibody structure or partial length antibody structure such as Fab

“Full length antibody” as used herein refers to an antibody having two full length antibody heavy chains and two full length antibody light chains. A full length antibody heavy chain (HC) consists of well known heavy chain variable and constant domains VH, CH1, CH2, and CH3. A full length antibody light chain (LC) consists of well known light chain variable and constant domains VL and CL. The full length antibody may be lacking the C-terminal lysine (K) in either one or both heavy chains.

The term “Fab-arm” or “half molecule” refers to one heavy chain-light chain pair that specifically binds an antigen.

Full length bispecific antibodies of the invention may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Intl. Publ. No. WO 2006/028936) may be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface may be used, as described in US Pat. Publ. No. US2010/0015133; US Pat. Publ. No. US2009/0182127; US Pat. Publ. No. US2010/028637 or US Pat. Publ. No. US2011/0123532. In other strategies, heterodimerization may be promoted by following substitutions (expressed as modified position in the first CH3 domain of the first heavy chain/modified position in the second CH3 domain of the second heavy chain): L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in U.S. Pat. Publ. No. US2012/0149876 or U.S. Pat. Publ. No. US2013/0195849

TABLE 1
Exemplary Amino Acid Sequences

| SEQ ID | | |
|---|---|---|
| NO: | Name | Amino Acid Sequence |
| SEQ ID | Common heavy chain | GFTFSSYA |
| NO: 1 | CDR-H1 | |
| SEQ ID | Common heavy chain | ISGSGGST |
| NO: 2 | CDR-H2 | |
| SEQ ID | Common heavy chain | AKSYGAFDY |
| NO: 3 | CDR-H3 | |
| SEQ ID | Common heavy chain | EVQLLESGGGLVQPGGSLRLSCAASGFTFSSYAMSWVRQAPGKGLEWVSA |
| NO: 4 | VH | ISGSGGSTYYADSVKGRFTISRDNSKNTLYLQMNSLRAEDTAVYYCAKSY |
| | | GAFDYWGQGTLVTVSS |
| SEQ ID | Common heavy chain | EVQLLESGGGLVQPGGSLRLSCAASGFTFSSYAMSWVRQAPGKGLEWVSA |
| NO: 5 | HC (wild-type) | ISGSGGSTYYADSVKGRFTISRDNSKNTLYLQMNSLRAEDTAVYYCAKSY |
| | | GAFDYWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICN |
| | | VNHKPSNTKVDKRVEPKSCDKTHTCPPCPAPELLGGPSVFLFPPKPKDTL |
| | | MISRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVYTL |
| | | PPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPG |
| SEQ ID | Common heavy chain | EVQLLESGGGLVQPGGSLRLSCAASGFTFSSYAMSWVRQAPGKGLEWVSA |
| NO: 6 | HC (with LALA | ISGSGGSTYYADSVKGRFTISRDNSKNTLYLQMNSLRAEDTAVYYCAKSY |
| | mutation) | GAFDYWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICN |
| | | VNHKPSNTKVDKRVEPKSCDKTHTCPPCPAPEAAGGPSVFLFPPKPKDTL |
| | | MISRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVYTL |
| | | PPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPG |
| SEQ ID | Common heavy chain | EVQLLESGGGLVQPGGSLRLSCAASGFTFSSYAMSWVRQAPGKGLEWVSA |
| NO: 7 | HC (with LALAPA | ISGSGGSTYYADSVKGRFTISRDNSKNTLYLQMNSLRAEDTAVYYCAKSY |
| | mutation) | GAFDYWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICN |
| | | VNHKPSNTKVDKRVEPKSCDKTHTCPPCPAPEAAGGPSVFLFPPKPKDTL |
| | | MISRTPEVTCVVVDVSHEDPEVKFNWYVDGVEVHNAKTKPREEQYNSTYR |
| | | VVSVLTVLHQDWLNGKEYKCKVSNKALAAPIEKTISKAKGQPREPQVYTL |
| | | PPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPG |
| SEQ ID | CEA binding part | SSNIANGI |
| NO: 8 | AC22 CDR-L1 | |
| SEQ ID | CEA binding part | FDN |
| NO: 9 | AC22 CDR-L2 | |
| SEQ ID | CEA binding part | GTWDFSYGIV |
| NO: 10 | AC22 CDR-L3 | |
| SEQ ID | CEA binding part | SSNIGIGL |
| NO: 11 | AC84 CDR-L1 | |
| SEQ ID | CEA binding part | NVN |
| NO: 11 | AC84 CDR-L2 | |
| SEQ ID | CEA binding part | GTWDFSYRVD |
| NO: 13 | AC84 CDR-L3 | |
| SEQ ID | CEA binding part | QSVLTQPPSVSAAPGQKVTISCSGSSSNIANGIVSWYQQLPGTAPKLLIY |
| NO: 14 | AC22 VL (light | FDNLRPSGIPDRFSGSKSGTSATLGITGLQTGDEADYYCGTWDFSYGIVF |
| | chain variable | GGGTKLTVL |
| | region) | |
| SEQ ID | CEA binding part | QSVLTQPPSVSAAPGQKVTISCSGSSSNIGIGLVSWYQQLPGTAPKLLIY |
| NO: 15 | AC84 VL (light | NVNTRPSGIPDRFSGSKSGTSATLGITGLQTGDEADYYCGTWDFSYRVDF |
| | chain variable | GGGTKLTVL |
| | region) | |
| SEQ ID | CEA binding part | QSVLTQPPSVSAAPGQKVTISCSGSSSNIANGIVSWYQQLPGTAPKLLIY |
| NO: 16 | AC22 LC (light | FDNLRPSGIPDRFSGSKSGTSATLGITGLQTGDEADYYCGTWDFSYGIVF |
| | chain) | GGGTKLTVLGQPKAAPSVTLFPPSSEELQANKATLVCLISDFYPGAVTVA |
| | | WKADSSPVKAGVETTTPSKQSNNKYAASSYLSLTPEQWKSHRSYSCQVTH |
| | | EGSTVEKTVAPTECS |
| SEQ ID | CEA binding part | QSVLTQPPSVSAAPGQKVTISCSGSSSNIGIGLVSWYQQLPGTAPKLLIY |
| NO: 17 | AC84 LC (light | NVNTRPSGIPDRFSGSKSGTSATLGITGLQTGDEADYYCGTWDFSYRVDF |
| | chain) | GGGTKLTVLGQPKAAPSVTLFPPSSEELQANKATLVCLISDFYPGAVTVA |
| | | WKADSSPVKAGVETTTPSKQSNNKYAASSYLSLTPEQWKSHRSYSCQVTH |
| | | EGSTVEKTVAPTECS |
| SEQ ID | CD28 binding part | QSVLYSSNNKNY |
| NO: 18 | AI3 CDR-L1 | |
| SEQ ID | CD28 binding part | WAS |
| NO: 19 | AI3 CDR-L2 | |
| SEQ ID | CD28 binding part | QQNLRPPET |
| NO: 20 | AI3 CDR-L3 | |
| SEQ ID | CD28 binding part | QSVLYSSNNKNY |
| NO: 21 | AI13 CDR-L1 | |
| SEQ ID | CD28 binding part | WAS |
| NO: 22 | AI13 CDR-L2 | |
| SEQ ID | CD28 binding part | QQNFRPPET |
| NO: 23 | AI13 CDR-L3 | |
| SEQ ID | CD28 binding part | DIVMTQSPDSLAVSLGERATINCKSSQSVLYSSNNKNYLAWYQQKPGQPP |
| NO: 24 | AI3 VL (light chain | KLLIYWASTRESGVPDRFSGSGSGTDFTLTISSLQAEDVAVYYCQQNLRP |
| | variable region) | PETFGQGTKVEIK |
| SEQ ID | CD28 binding part | DIVMTQSPDSLAVSLGERATINCKSSQSVLYSSNNKNYLAWYQQKPGQPP |
| NO: 25 | AI13 VL (light | KLLIYWASTRESGVPDRFSGSGSGTDFTLTISSLQAEDVGVYYCQQNFRP |
| | chain variable | PETFGGGTKVEIK |
| | region) | |
| SEQ ID | CD28 binding part | DIVMTQSPDSLAVSLGERATINCKSSQSVLYSSNNKNYLAWYQQKPGQPP |
| NO: 26 | AI3 LC (light | KLLIYWASTRESGVPDRFSGSGSGTDFTLTISSLQAEDVAVYYCQQNLRP |
| | chain) | PETFGQGTKVEIKRTVAAPSVFIFPPSDEQLKSGTASVVCLLNNFYPREA |
| | | KVQWKVDNALQSGNSQESVTEQDSKDSTYSLSSTLTLSKADYEKHKVYAC |
| | | EVTHQGLSSPVTKSFNRGEC |
| SEQ ID | CD28 binding part | DIVMTQSPDSLAVSLGERATINCKSSQSVLYSSNNKNYLAWYQQKPGQPP |
| NO: 27 | AI13 LC (light | KLLIYWASTRESGVPDRFSGSGSGTDFTLTISSLQAEDVGVYYCQQNFRP |
| | chain) | PETFGGGTKVEIKRTVAAPSVFIFPPSDEQLKSGTASVVCLLNNFYPREA |
| | | KVQWKVDNALQSGNSQESVTEQDSKDSTYSLSSTLTLSKADYEKHKVYAC |
| | | EVTHQGLSSPVTKSFNRGEC |

Table 2 shows exemplary bispecific antibodies. Nomenclature for each antibodies are shown and the individual kappa light chain, lambda light chain and heavy chain regions of the KA-Bodies of the disclosure are described. “AI” denotes anti-Human CD28 antigen binding regions, “AC” denotes Anti-human CEA antigen binding regions, “N denotes a heavy chain with LALAPA mutation. Alternate nomenclature is shown within the brackets.

TABLE 2
Exemplary Bispecific Antibodies

| | Kappa light | Lambda light | |
|---|---|---|---|
| BsAb name | chain | chain | Heavy chain |
| AI3AC84/N | AI3 (AI_1H5) | AC84 | Common VH |
| AI13AC84/N | AI13 (AI_2H10) | (AC1g6_1F1) | with LALAPA |
| | | | mutation |

### PD-1

PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers.

The programmed death 1 (PD-1) receptor and PD-1 ligands 1 and 2 (PD-L1 and PD-LA, respectively) play integral roles in immune regulation. Expressed on activated T cells, PD-1 is activated by PD-L1 (also known as B7-H1) and PD-L2 expressed by stromal cells, tumor cells, or both, initiating T-cell death and localized immune suppression potentially providing an immune-tolerant environment for tumor development and growth. Conversely, inhibition of this interaction can enhance local T-cell responses and mediate antitumor activity. Specifically, antibodies targeting programmed cell death protein-1 (PD-1) or its ligand PD-L1 rescue T cells from exhausted status and revive immune response against cancer cells.

### Anti-PD-1 Antibodies

Anti-PD-1 antibodies that bind specifically to PD-1 and block binding to its ligands PD-L1 or PD-L2 are well known in the art.

For example, huMAbs that bind specifically to PD-1 with high affinity have been disclosed in U.S. Pat. Nos. 8,008,449 and 8,779,105. Other anti-PD-1 mAbs have been described in, for example, U.S. Pat. Nos. 6,808,710, 7,488,802, 8, 168,757 and 8,354,509, and PCT Publication No. WO 2012/145493. Each of the anti-PD-1 HuMAbs disclosed in U.S. Pat. No. 8,008,449 has been demonstrated to exhibit one or more of the following characteristics: (a) binds to human PD-1 with a KD of 1×10-7 M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates Ab responses; and (j) inhibits tumor cell growth in vivo. Anti-PD-1 Abs usable in the present invention include mAbs that bind specifically to human PD-1 and exhibit at least one, preferably at least five, of the preceding characteristics.

In one embodiment, the anti-PD-1 Ab is nivolumab. Nivolumab (also known as “OPDIVO®”; formerly designated 5C4, BMS-936558, MDX-1106, or ONO-4538) is a fully human IgG4 (S228P) PD-1 immune checkpoint inhibitor Ab that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2 (9): 846-56).

In another embodiment, the anti-PD-1 Ab is pembrolizumab. Pembrolizumab (also known as “KEYTRUDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587; see also www.cancer.gov/drugdictionary?cdrid=695789 (last accessed: Dec. 14, 2014).

In other embodiments, the anti-PD-1 Ab is MEDI0608 (formerly AMP-514), which is a monoclonal antibody. MEDI0608 is described, for example, in U.S. Pat. No. 8,609,089B2 or in www.cancer.gov/drugdictionary?cdrid=756047 (last accessed Dec. 14, 2014).

In some embodiments, the anti-PD-1 antibody is Pidilizumab (CT-011), which is a humanized monoclonal antibody. Pidilizumab is described in U.S. Pat. No. 8,686,119 B2 or WO 2013/014668 A1.

Other anti-PD-1 antibodies include for example, cemiplimab, sintilimab, camrelizumab, toripalimab, tislelizumab, zimberelimab, prolgolimab, and dostarlimab.

Anti-PD-1 Abs usable in the disclosed methods also include isolated Abs that bind specifically to human PD-1 and cross-compete for binding to human PD-1 with nivolumab (see, e.g., U.S. Pat. Nos. 8,008,449 and 8,779,105; WO 2013/173223, pembrolizumab, cemiplimab, sintilimab, camrelizumab, toripalimab, tislelizumab, zimberelimab, prolgolimab. and dostarlimab). The ability of Abs to cross-compete for binding to an antigen indicates that these Abs bind to the same epitope region of the antigen and sterically hinder the binding of other cross-competing Abs to that particular epitope region. Cross-competing Abs can be readily identified based on their ability to cross-compete in standard PD-1 binding assays such as Biacore analysis, ELISA assays or flow cytometry (see, e.g., WO 2013/173223).

Anti-PD-1 Abs usable in the methods of the disclosed invention also include antigen-binding portions of the above Abs. It has been amply demonstrated that the antigen-binding function of an Ab can be performed by fragments of a full-length Ab. Examples of binding fragments encompassed within the term “antigen-binding portion” of an Ab include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; and (iv) a Fv fragment consisting of the VL and VI/domains of a single arm of an Ab.

### Methods of Use

Therapeutic formulations of the invention, which include the CEAxCD28 bsAbs and a PD-1 blocking antibody are used to treat or alleviate a symptom associated with a cancer, such as, by way of non-limiting example, leukemias, lymphomas, breast cancer, colon cancer, ovarian cancer, bladder cancer, prostate cancer, glioma, lung & bronchial cancer, colorectal cancer, pancreatic cancer, esophageal cancer, liver cancer, urinary bladder cancer, kidney and renal pelvis cancer, oral cavity & pharynx cancer, uterine corpus cancer, and/or melanoma. The present invention also provides methods of treating or alleviating a symptom associated with a cancer. A therapeutic regimen is carried out by identifying a subject, e.g., a human patient suffering from (or at risk of developing) a cancer, using standard methods. The cancer is comprised of tumor cells that express CEA and PD-L1 and/or PD-L2 is detected in cells withing the tumor microenvironment. Cells within the tumor microenvironment include but are not limited to tumor cells.

The CEAxCD28 bsAb and a PD-1 blocking antibody can be administered simultaneously. Alternatively, the CEAxCD28 bsAb is administered before PD-1 blocking antibody. The CEAxCD28 bsAb is administered after the PD-1 blocking antibody. In some aspects the administration of CEAxCD28 bsAb and a PD-1 blocking antibody are alternated in accordance with a multi-dosing regimen.

Efficaciousness of treatment is determined in association with any known method for diagnosing or treating the particular immune-related disorder. Alleviation of one or more symptoms of the immune-related disorder indicates that the antibody confers a clinical benefit.

### Pharmaceutical Compositions

The antibodies of the invention (also referred to herein as “active compounds”), and derivatives, fragments, analogs and homologs thereof, can be incorporated into pharmaceutical compositions suitable for administration. Such compositions typically comprise the antibody and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

### Definitions

As used herein, including the appended claims, the singular forms of words such as “a,” “an,” and “the,” include their corresponding plural references unless the context clearly dictates otherwise.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D})). Affinity can be measured by common methods known in the art, including KinExA and Biacore.

As used herein, the term “antibody” includes, but is not limited to, monoclonal antibodies, polyclonal antibodies, multispecific antibodies (e.g., bispecific antibodies), fully human antibodies, and chimeric antibodies.

As used herein, unless otherwise indicated, “antigen-binding fragment” refers to antigen-binding fragments of antibodies, i.e. antibody fragments that retain the ability to bind to the antigen bound by the full-length antibody, e.g. fragments that retain one or more CDR regions. Examples of antibody binding fragments include, but are not limited to, Fab, Fab′, F(ab′)₂, Fv fragments and individual antibody heavy chains or light chains, and individual heavy chain or light chain variable regions.

A “Fab fragment” is comprised of one light chain and the CH1 and variable regions of one heavy chain. The heavy chain of a Fab molecule cannot form a disulfide bond with another heavy chain molecule. A “Fab fragment” can be the product of papain cleavage of an antibody.

A “Fc” region contains two heavy chain fragments comprising the CH2 and CH3 domains of an antibody. The two heavy chain fragments are held together by two or more disulfide bonds and by hydrophobic interactions of the CH3 domains.

A “Fab′ fragment” contains one light chain and a portion or fragment of one heavy chain that contains the VH domain and the CH1 domain and also the region between the CH1 and CH2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the CH1 and CH2 domains, such that an interchain disulfide bond is formed between the two heavy chains. A F(ab′)₂ fragment thus is composed of two Fab′ fragments that are held together by a disulfide bond between the two heavy chains. An “F(ab′)₂ fragment” can be the product of pepsin cleavage of an antibody. The “Fv region” comprises the variable regions from both the heavy and light chains but lacks the constant regions.

“Isolated antibody” refers to the purification status and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

The term “monoclonal antibody”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains that are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example. See also Presta (2005) J. Allergy Clin. Immunol. 116:731.

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody that comprises mouse immunoglobulin sequences only. Alternatively, a fully human antibody may contain rat carbohydrate chains if produced in a rat, in a rat cell, or in a hybridoma derived from a rat cell. Similarly, “rat antibody” refers to an antibody that comprises rat immunoglobulin sequences only.

In general, the basic “antibody” structural unit comprises a tetramer. In a monospecific antibody, each tetramer includes two identical pairs of polypeptide chains, each pair having one “light” (about 25 kDa) and one “heavy” chain (about 50-70 kDa). The amino-terminal portion of each chain includes a “variable region” or “variable domain” of about 100 to 110 or more amino acids primarily responsible for antigen recognition. The carboxy-terminal portion of the heavy chain may define a constant region primarily responsible for effector function.

Typically, human constant light chains are classified as kappa and lambda light chains. Furthermore, human constant heavy chains are typically classified as mu, delta, gamma, alpha, or epsilon, and define the antibody's isotype as IgM, IgD, IgG, IgA, and IgE, respectively. Subtypes of these IgG include, for example, IgG1 and IgG4.

“Variable region,” “variable domain,” “V region,” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “VII.” The variable region of the light chain may be referred to as “V_{L}.” Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody Vu beta-sheet framework, or one of three hypervariable regions (L1, L2, or L3) within the non-framework region of the antibody V_{L} beta.-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved .beta.-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

Sequence identity refers to the degree to which the amino acids of two polypeptides are the same at equivalent positions when the two sequences are optimally aligned.

Sequence similarity includes identical residues and non-identical, biochemically related amino acids. Biochemically related amino acids that share similar properties and may be interchangeable are discussed above.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity.

The term “epitope,” as used herein, refers to an area or region on an antigen to which an antibody or antigen-binding fragment binds. Binding of an antibody or antigen-binding fragment thereof disclosed herein to an epitope means that the antibody or antigen-binding fragment thereof binds to one or more amino acid residues within the epitope.

“Isolated” nucleic acid molecule or polynucleotide means a DNA or RNA, e.g., of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a polynucleotide comprising” (or the like) a particular nucleotide sequence does not encompass intact chromosomes. Isolated polynucleotides “comprising” specified nucleic acid sequences may include, in addition to the specified sequences, coding sequences for up to ten or even up to twenty or more other proteins or portions or fragments thereof or may include operably linked regulatory sequences that control expression of the coding region of the recited nucleic acid sequences, and/or may include vector sequences.

The phrase “control sequences” refers to polynucleotide sequences necessary or helpful for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers. In an embodiment of the invention, the polynucleotide is operably linked to a promoter such as a viral promoter, a CMV promoter, an SV40 promoter or a non-viral promoter or an elongation factor (EF)-1 promotor; and/or an intron.

A nucleic acid is “operably linked” when it is placed into a functional relationship with another polynucleotide. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, but not always, “operably linked” means that the polynucleotide sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

Host cells include eukaryotic and prokaryotic host cells, including mammalian cells. Host cells include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells and HEK-293 cells. Mammalian host cells include human, mouse, rat, dog, monkey, pig, goat, bovine, horse and hamster cells. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia pastoris, Pichia finlandica, Pichia trehalophia, Pichia koclamae, Pichia membranaefaciens, Pichia minuta (Ogataea minuta, Pichia lindnen), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Pichia sp., any Saccharomyces sp., Hansenula polymorpha, any Kluyveromyces sp., Candida albicans, any Aspergillus sp., Trichoderma reesei, Chrysosporium lucknowense, any Fusarium sp., Yarrowia lipolytica, and Neurospora crassa. The present invention includes any host cell (e.g., a CHO cell or Pichia cell, e.g., Pichia pastoris) containing an anti-ILT4 antibody or antigen-binding fragment thereof or containing a polynucleotide encoding such an antibody or fragment or containing a vector that contains the polynucleotide.

“Treat” or “treating” means to administer antibodies or antigen-binding fragments thereof of the present invention, to a subject having one or more symptoms of a disease for which the antibodies and antigen-binding fragments are effective, e.g., in the treatment of a subject having cancer or an infectious disease, or being suspected of having cancer or infectious disease, for which the agent has therapeutic activity. Typically, the antibody or fragment is administered in an “effective amount” or “effective dose” which will alleviate one or more symptoms (e.g., of cancer or infectious disease) in the treated subject or population, whether by inducing the regression or elimination of such symptoms or by inhibiting the progression of such symptom(s), e.g., cancer symptoms such as tumor growth or metastasis, by any clinically measurable degree. The effective amount of the antibody or fragment may vary according to factors such as the disease stage, age, and weight of the patient, and the ability of the drug to elicit a desired response in the subject.

“Programmed Death-1 (PD-1)” refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No U64863.

“Programmed Death Ligand-1 (PD-L1)” is one of two cell surface glycoprotein ligands for PD-1 (the other being PD-L2) that downregulate T cell activation and cytokine secretion upon binding to PD-1. The term “PD-L1” as used herein includes human PD-L1 (bPD-L1), variants, isoforms, and species homologs of hPD-L1, and analogs having at least one common epitope with hPD-L1. The complete hPD-L1 sequence can be found under GenBank Accession No. Q9NZQ7.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in the severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the term “subtherapeutic dose” or “subtherapeutically effective amount” means a dose of a therapeutic compound (e.g., an antibody) that is lower than the usual or typical dose of the therapeutic compound when administered alone for the treatment of cancer.

By way of example for the treatment of tumors, a therapeutically effective amount of an anti-cancer agent preferably inhibits cell growth or tumor growth by at least about 20%. more preferably by at least about 40%, even more preferably by at least about 60%, and still more preferably by at least about 80% relative to untreated subjects. In other embodiments of the invention, tumor regression may be observed and continue for a period of at least about 20 days, more preferably at least about 40 days, or even more preferably at least about 60 days.

### EXAMPLES

### Example 1: In Vivo Assessment of the Synergy Between CEAxCD28 bsAbs and PD-1 Blockade

Combination of A13AC84 N with PD-1 Blockade in the Treatment of MC38-hCEA Tumors Engrafted in CD28-Humanized Mice

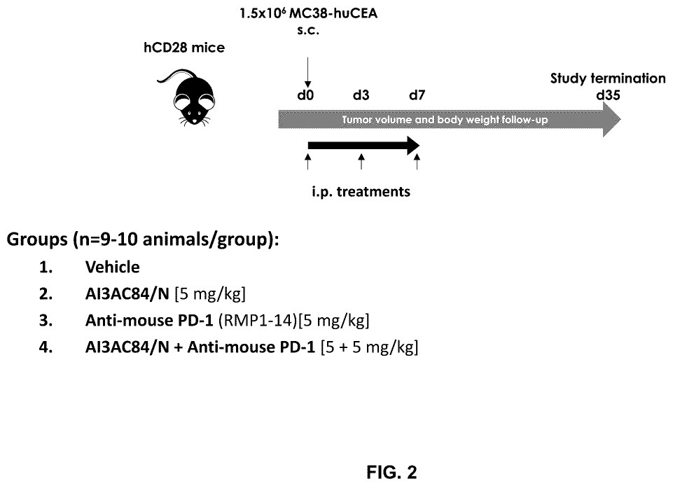

This efficacy study aimed at assessing the synergy between AI3AC84/N and a blocking PD-1 antibody in vivo. The experimental design is summarized in FIG. 2. Briefly: MC38-huCEA cells (MC38 cells expressing human CEA at the cell surface) were engrafted subcutaneously in huCD28 mice (mice in which the CD28 extracellular domain is entirely humanized, with physiological regulation and expression pattern of the human CD28, coupled to a fully functional mouse immune system; genOway). Mice were randomized on day 0 based on their body weight and were treated for a total of 3 times intraperitoneally (i.p.) on day 0, 3 and 7 with: vehicle, 5 mg/kg RMP1-14 (a blocking rat anti-mouse PD-1 mAb; Bio X Cell), 5 mg/kg AI3AC84/N (CEAxCD28 of the present invention), or a combination of both RMP1-14 and AI3AC84/N, each at 5 mg/kg. Tumors were measured three times per week by digital caliper until the endpoint of the experiment (tumor volume=1500 mm3). Tumor volume was calculated using the formula (length×width²)×0.5.

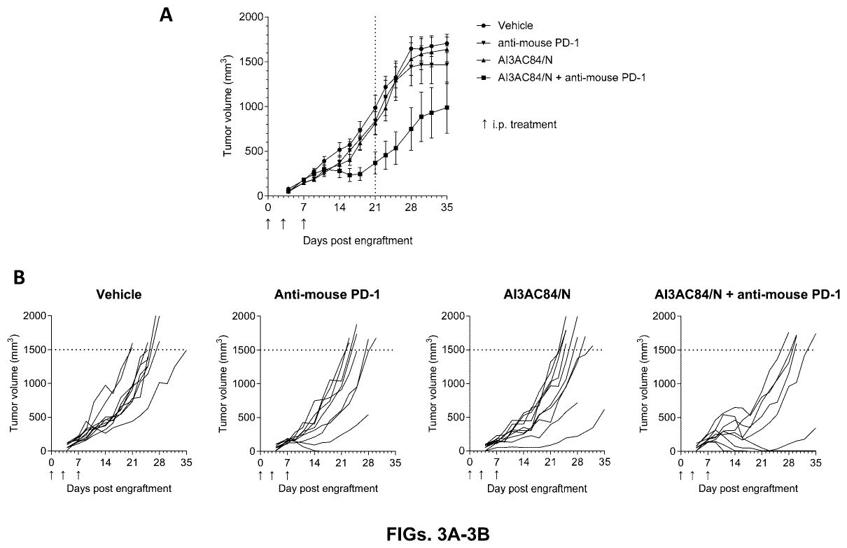

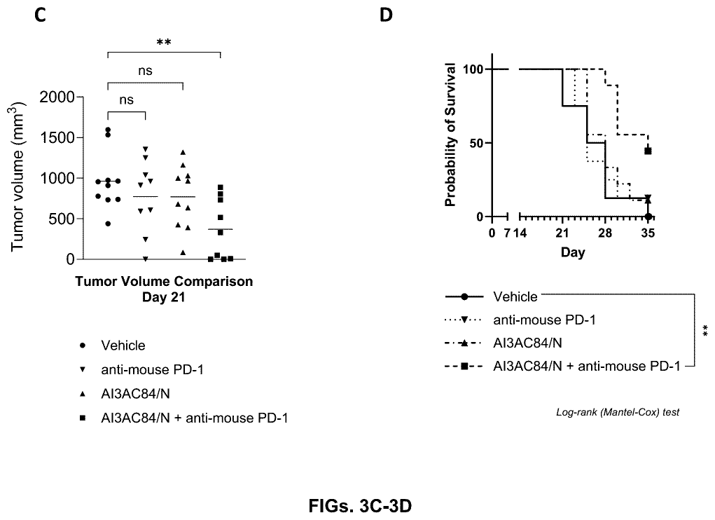

Only a mild, non-statistically significant inhibition of tumor growth over the vehicle group was observed in mice treated with RMP1-14 or AI3AC84/N as single agent (FIG. 3A+B). However, AI3AC84/N combined with the blockade of PD-1 mediated by the RMP1-14 antibody, induced a significant delay in tumor growth (FIG. 3A), with 3 out of 9 mice considered tumor-free at study termination (FIG. 3B). Tumor volume comparison at day 21, when the first vehicle mice reached the tumor endpoint, highlights a statistical anti-tumor activity of the combination treatment (FIG. 3C), which led to an overall survival advantage (FIG. 3D).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a cancer in a subject comprising administering to the subject a CEAxCD28 bispecific antibody and a blocking anti-PD-1 antibody.

2. The method of claim 1, wherein the CEAxCD28 bispecific antibody comprises:
a) a CEA antigen binding domain comprising:
i) a heavy chain variable region having a complementarity determining region 1 (CDRH1) comprising the amino acid sequence of SEQ ID NO: 1; a complementarity determining region 2 (CDRH2) comprising the amino acid sequence of SEQ ID NO: 2; and a complementarity determining region 3 (CDRH3) comprising the amino acid sequence of SEQ ID NO: 3; and
ii) a first light chain variable region having a complementarity determining region 1 (CDRL1) comprising the amino acid sequence of SEQ ID NO: 11;
a complementarity determining region 2 (CDRL2) comprising the amino acid sequence of NVN; and a complementarity determining region 3 (CDRL3) comprising the amino acid sequence of SEQ ID NO: 13; and
b) a CD28 binding domain comprising:
i) a heavy chain variable region having a complementarity determining region 1 (CDRH1) comprising the amino acid sequence of SEQ ID NO: 1; a complementarity determining region 2 (CDRH2) comprising the amino acid sequence of SEQ ID NO: 2; and a complementarity determining region 3 (CDRH3) comprising the amino acid sequence of SEQ ID NO: 3; and
ii) a first light chain variable region having a complementarity determining region 1 (CDRL1) comprising the amino acid sequence of SEQ ID NO: 18; a complementarity determining region 2 (CDRL2) comprising the amino acid sequence of WAS; and a complementarity determining region 3 (CDRL3) comprising the amino acid sequence of SEQ ID NO: 20.

3. The method of claim 2, wherein:
a) the CEA antigen binding domain comprises a variable light chain comprising the amino acid sequence of SEQ ID: 15; and a variable heavy chain comprising the amino acid sequence of SEQ ID: 4; and
b) the CD28 antigen binding domain comprises a variable light chain comprising the amino acid sequence of SEQ ID: 24; and a variable heavy chain comprising the amino acid sequence of SEQ ID: 4.

4. The method of claim 2 wherein:
a) the CEA antigen binding domain comprises a light chain comprising the amino acid sequence of SEQ ID: 17; and a heavy chain comprising the amino acid sequence of SEQ ID: 7; and
b) the CD28 antigen binding domain comprises a light chain comprising the amino acid sequence of SEQ ID: 26; and a heavy chain comprising the amino acid sequence of SEQ ID: 4.

5. The method of claim 1, wherein the blocking anti-PD-1 antibody is selected from the group comprising nivolumab, pembrolizumab, cemiplimab, sintilimab, camrelizumab, toripalimab, tislelizumab, zimberelimab, prolgolimab, and dostarlimab.

6. The method of claim 1, wherein the cancer is selected from the group comprising leukemias, lymphomas, breast cancer, colon cancer, ovarian cancer, bladder cancer, prostate cancer, glioma, lung & bronchial cancer, colorectal cancer, pancreatic cancer, esophageal cancer, liver cancer, urinary bladder cancer, kidney and renal pelvis cancer, oral cavity & pharynx cancer, uterine corpus cancer, and melanoma.

7. The method of claim 1, wherein the bispecific antibody and the anti-PD-1 antibody are administered simultaneously.

8. The method of claim 1, wherein the bispecific antibody is administered prior to anti-PD-1 antibody.

9. The method of claim 1, wherein the bispecific antibody is administered after the anti-PD-1 antibody.

10. The method of claim 1, wherein the CEAxCD28 bispecific antibody and/or the blocking anti-PD-1 antibody are administered intravenously.

11. The method of claim 1, wherein the subject is human.

12. The method of claim 1, where the treatment reduces the proliferation of the cancer cell or increases the killing of a cancer cell.

13. The method of claim 1, wherein the treatment enables tumor-specific T cell activation.

14. The method of claim 1, wherein administration of the blocking anti-PD-1 increases the anti-tumor activity of the bispecific antibody compared to when the bispecific antibody is administered as a single therapeutic agent.

15. A formulation comprising a CEAxCD28 bispecific antibody and a blocking anti-PD-1 antibody.

16. The formulation of claim 15, wherein the CEAxCD28 bispecific antibody comprises:
a) a CEA antigen binding domain comprising:
i) a heavy chain variable region having a complementarity determining region 1 CDRH1 comprising the amino acid sequence of SEQ ID NO: 1; a complementarity determining region 2 (CDRH2) comprising the amino acid sequence of SEQ ID NO: 2; and a complementarity determining region 3 (CDRH3) comprising the amino acid sequence of SEQ ID NO: 3; and
ii) a first light chain variable region having a complementarity determining region 1 (CDRL1) comprising the amino acid sequence of SEQ ID NO: 11;
a complementarity determining region 2 (CDRL2) comprising the amino acid sequence of NVN; and a complementarity determining region 3 (CDRL3) comprising the amino acid sequence of SEQ ID NO: 13;
b) a CD28 binding domain comprising:
i) a heavy chain variable region having a complementarity determining region 1 (CDRH1) comprising the amino acid sequence of (SEQ ID NO: 1); a complementarity determining region 2 (CDRH2) comprising the amino acid sequence of (SEQ ID NO: 2); and a complementarity determining region 3 (CDRH3) comprising the amino acid sequence of (SEQ ID NO: 3); and
ii) a first light chain variable region having a complementarity determining region 1 (CDRL1) comprising the amino acid sequence of SEQ ID NO: 18; a complementarity determining region 2 (CDRL2) comprising the amino acid sequence of WAS; and a complementarity determining region 3 (CDRL3) comprising the amino acid sequence of SEQ ID NO: 20; and
c) the PD-1 blocking antibody is selected from the group consisting of nivolumab, pembrolizumab, cemiplimab, sintilimab, camrelizumab, toripalimab, tislelizumab, zimberelimab, prolgolimab, and dostarlimab.

17. A kit comprising:
a) a formulation suitable for human administration comprising a CEAxCD28 bispecific antibody comprising:
i) a CEA antigen binding domain comprising:
a) a heavy chain variable region having a complementarity determining region 1 CDRH1 comprising the amino acid sequence of SEQ ID NO: 1; a complementarity determining region 2 (CDRH2) comprising the amino acid sequence of SEQ ID NO: 2; and a complementarity determining region 3 (CDRH3) comprising the amino acid sequence of SEQ ID NO: 3; and
b) a first light chain variable region having a complementarity determining region 1 (CDRL1) comprising the amino acid sequence of SEQ ID NO: 11; a complementarity determining region 2 (CDRL2) comprising the amino acid sequence of NVN; and a complementarity determining region 3 (CDRL3) comprising the amino acid sequence of SEQ ID NO: 13;
ii) a CD28 binding domain comprising:
a) a heavy chain variable region having a complementarity determining region 1 (CDRH1) comprising the amino acid sequence of SEQ ID NO: 1; a complementarity determining region 2 (CDRH2) comprising the amino acid sequence of SEQ ID NO: 2; and a complementarity determining region 3 (CDRH3) comprising the amino acid sequence of SEQ ID NO: 3; and
b) a first light chain variable region having a complementarity determining region 1 (CDRL1) comprising the amino acid sequence of SEQ ID NO: 18;
a complementarity determining region 2 (CDRL2) comprising the amino acid sequence of WAS; and a complementarity determining region 3 (CDRL3) comprising the amino acid sequence of SEQ ID NO: 20; and
b) a formulation suitable for human administration comprising a PD-1 blocking antibody is selected from the group consisting of nivolumab, pembrolizumab, cemiplimab, sintilimab, camrelizumab, toripalimab, tislelizumab, zimberelimab, prolgolimab, and dostarlimab; and
c) instructions for use.
